(19) United States
(12) Patent Application Publication
(10) Pub. No.: US 2011/0126846 A1
(43) Pub. Date: Jun. 2, 2011

(54) Original ear popper stopper pressurized headset

(76) Inventor: Ralph P. Hill, Chicago, IL (US)

(21) Appl. No.: 12/927,289

(22) Filed: Nov. 10, 2010

Related U.S. Application Data
(60) Provisional application No. 61/281,086, filed on Nov. 12, 2009.

(51) Int. Cl. A61F 11/14

(52) U.S. Cl. 128/867

(57) ABSTRACT

The present invention device provides a device designed to prevent ear pain or discomfort due to air pressure changes when flying, comprising a head unit resembling a set of lightweight headphones, featuring an adjustable bridge that allows the unit to be worn by adults and children alike. The unique aspects of this product are found in its dual ear cover housings. Each circular covering features a comfortable, padded exterior to fit over the ears; directly behind the padding is a small section of empty space. Extending inward, an area of padded foam provides a blocking agent for air as well as noise. Further helping to protect the ears from changes in air pressure, each side of the Original Ear Popper Stopper is lined with bubble wrap, offering even more insulation.

### CLAIM OF PRIORITY

This patent application claims priority under 35 USC 119 (e) (1) from U.S. Provisional Patent Application Ser. No. 61/281,086 filed Nov. 12, 2009, of common inventorship herewith entitled, “The Original Ear Popper Stopper Pressurized Headset”

### FIELD OF THE INVENTION

The present invention pertains to the field of ear pressure relief, and more specifically to the field of prevention of preventing ear pressure buildup when traveling by airplane, such as when in the course of flying as a passenger of an airplane.

### BACKGROUND OF THE INVENTION

The prior art has put forth several designs for ear pressure regulators. Among these are:

U.S. Pat. No. 5,024,612 to van den Honert describes a tinnitus suppression device.

U.S. Pat. No. 5,467,784 to Mobley describes a porous pressure regulating earplug.

U.S. Pat. No. 5,819,745 to Mobley describes a slow leak rate pressure regulating earplug.

U.S. Pat. No. 6,129,086 to Gzybowski describes a manually regulated device for adjusting ear pressure.

None of these prior art references describe the present invention.

### SUMMARY OF THE INVENTION

It is an object of the present invention to provide a device for relief to an individual experiencing ear discomfort or pain associated with changes in air pressure.

### BRIEF DESCRIPTION OF THE DRAWINGS

FIG. 1 is a front perspective view of the device of the present invention.

FIG. 2 is a schematic view of the ear cup configuration of the device of the present invention.

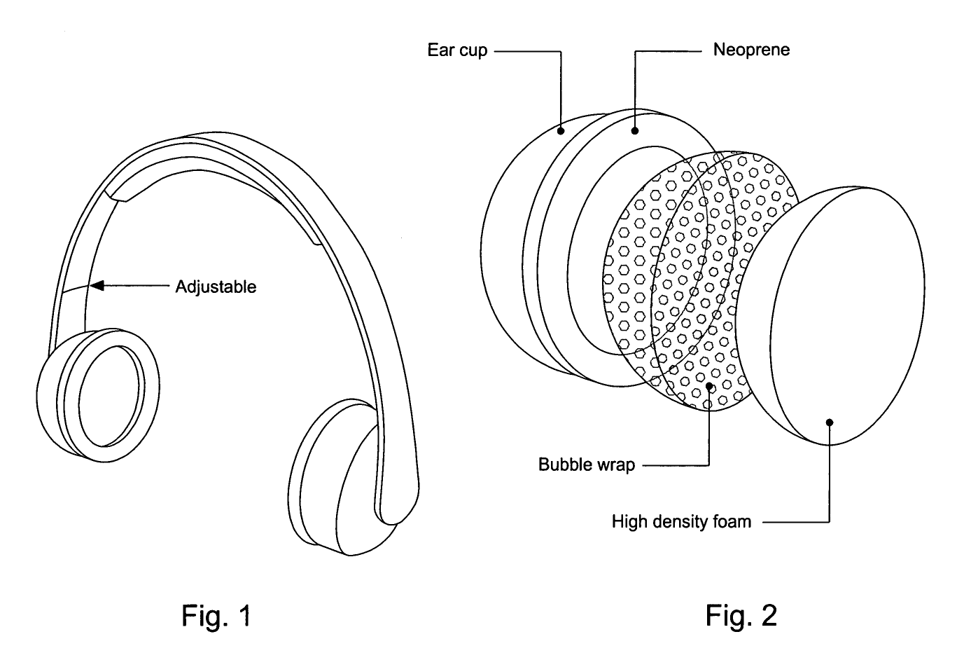

### DETAILED DESCRIPTION OF THE INVENTION

Air travel is generally one of the safest and most convenient methods of traveling. Millions of passengers around the world choose to travel by air each year, the vast majority of which experience no ill effects from their journey. However, there is one small malady that effects thousands of flyers each year: ear popping. In simplest terms, this occurs during the pressure changes in an airplane while in the air, and can be more intense as the plane ascends and descends. Technically speaking, the phenomenon is caused by a pocket of air inside the middle ear, the space behind the eardrum. The air in the middle ear is constantly being absorbed by the membranes that line the cavity, so the internal pressure can easily drop. Fortunately, air is frequently resupplied to the middle ear during the process of swallowing. Usually when a person swallows, a small bubble of air passes from the throat or back of the nose, through a narrow tube known as the Eustachian tube, into the middle ear. As it does this, it makes a tiny click or popping sound. This action keeps the air pressure on both sides of the eardrum about equal. If the air pressure isn't equal, the ear feels blocked. With pressure changes in a plane, the pocket of air in the middle ear is particularly vulnerable to the changes in air pressure as you go up in a plane. The higher the plane, the lower the air pressure around you, although inside the cabin of the airplane you're protected, to some extent, from these pressure changes. If the air in the middle ear can't be replenished fast enough, then pressure drops so low that a vacuum develops. The eardrum is sucked inwards in a stretched fashion and can't vibrate properly, so sounds become muffled. The eardrums can stretch when the Eustachian tube is blocked, as the result of a bad cold, for example, or a nasal allergy, or because it's narrow as a result of childhood ear infections.

The present invention is the Original Ear Popper Stopper, a helpful aid in warding off the “ear popping” phenomena on airplanes. Simply stated, this invention is a set of ear coverings specially designed for use while flying.

The Original Ear Popper Stopper resembles a set of lightweight headphones, featuring an adjustable bridge that allows the unit to be worn by adults and children alike. The unique aspects of this product are found in its dual ear cover housings. Each circular covering features a comfortable, padded exterior to fit over the ears; directly behind the padding is a small section of empty space. Extending inward, an area of padded foam provides a blocking agent for air as well as noise. Further helping to protect the ears from changes in air pressure, each side of the Original Ear Popper Stopper is lined with bubble wrap, offering even more insulation.

Simply designed yet effective in application, air passengers need only don a unit as soon as a flight attendant announces that the plane has been cleared for takeoff. In this manner, the present invention prevents the buildup of pressure in the Eustachian tubes, eliminating the uncomfortable popping sensation. Comfortably designed, the device of the present invention can be worn during the entire flight, or just put on during ascents and descents. With the Original Ear Popper Stopper in place, air passengers need not worry about the formation of headaches that can result from the ear popping phenomena. Moreover, this clever invention proves especially ideal for children who fly. Affordably priced, the Original Ear Popper Stopper will be well received by the vast majority of consumers who travel by air, whether for business or pleasure, a sizable market potential.

Although this invention has been described with respect to specific embodiments, it is not intended to be limited thereto and various modifications which will become apparent to the person of ordinary skill in the art are intended to fall within the spirit and scope of the invention as described herein taken in conjunction with the accompanying drawings and the appended claims.

1. A device designed to prevent ear pain or discomfort due to air pressure changes when flying, comprising a device having an adjustable bridge to fit over the head of a user and circular ear cover housings, wherein the ear coverings comprise a layered configuration comprising a comfortable, padded exterior to fit over the ears and a small section of empty space immediately behind each ear covering.

2. The device of claim 1, wherein the ear covering further comprises an additional layer, extending inward, of padded foam to provide a blocking agent for air as well as noise, and to further help to protect the ears from changes in air pressure, each side of the device is lined with bubble wrap, offering even more insulation.
